(19) United States
(12) Patent Application Publication
(10) Pub. No.: US 2022/0265279 A1
(43) Pub. Date: Aug. 25, 2022

(54) ANCHOR FOR EMBOLIC COILS, AND EMBOLIC COIL COMPRISING SAME

(71) Applicants: HANS BIOMED CORP, Seoul (KR); THE ASAN FOUNDATION, Seoul (KR); UNIVERSITY OF ULSAN FOUNDATION FOR INDUSTRY COOPERATION, Ulsan (KR)

(72) Inventors: Deok Hee LEE, Ulsan (KR); Hong Hee JUNG, Seoul (KR); Eu Gene LIH, Seoul (KR); Dae Hyung LEE, Gyeonggi-do (KR); Seung Hyun LEE, Gyeonggi-do (KR); Ye Ji CHOI, Seoul (KR); Mi Ri KIM, Seoul (KR); Yun Sun SONG, Seoul (KR); Seon Moon HWANG, Seoul (KR); Joon Ho CHOI, Seoul (KR); Tae Il KIM, Seoul (KR); Seng Yong CHUN, Seoul (KR); Jun Hyun KIM, Seoul (KR); Jun Young MAENG, Seoul (KR); Ga Young LEE, Seoul (KR)

(21) Appl. No.: 17/627,358

(22) PCT Filed: Jul. 17, 2020
(86) PCT No.: PCT/KR2020/009445

§ 371 Date: Jan. 14, 2022

(30) Foreign Application Priority Data
Jul. 17, 2019 (KR) 10-2019-0086632

(51) Int. Cl. A61B 17/12; A61L 31/02

(52) U.S. Cl. CPC A61B 17/12113; A61B 17/1215; A61L 31/022; A61B 2017/00867

(57) ABSTRACT

An anchor for an embolic coil, and an embolic coil including the same are provided. The anchor for an embolic coil includes a hollow main body and a central shaft arranged in the hollow main body, wherein the hollow main body is made of a braided composite filament including a polymer filament and a first alloy filament, and the central shaft includes a second alloy filament that is a material different from the first alloy filament.

### TECHNICAL FIELD

An anchor for an embolic coil and an embolic coil comprising same are disclosed. More specifically, an anchor for an embolic coil, capable of improving the lesion treatment effect of an embolic coil, and an embolic coil comprising same, are disclosed.

### BACKGROUND ART

Vascular disorders and defects, such as aneurysms and other arterio-venous malformations, are particularly difficult to treat when located in the vicinity of critical tissues or when rapid access to the malformations is not possible. The causes of these two difficulties are applied particularly to cranial aneurysms. Due to sensitive brain tissues surrounding the cranial blood vessels and limited access thereto, surgical treatment of defects in cranial blood vessel structures is very challenging and often dangerous.

Alternative treatments include vascular occlusion devices, such as embolic coils, deployed by using a catheter delivery system. In currently preferred procedures for treating cerebral aneurysms, the distal end of an embolic coil delivery catheter is inserted into the non-cranial vasculature of a patient, typically via the femoral artery in the groin, and then guided to a desired delivery site within the cranium. A number of delivery techniques for vascular-occlusion devices, including the use of fluid pressure to release an embolic coil once the embolic coil is correctly positioned, are disclosed in, for example, U.S. Pat. Nos. 6,063,100 and 6,179,857 by Diaz et al.

Multiple embolic coils of varying lengths, typically between 1 and 30 centimeters in length and preselected stiffness, are often sequentially packed within a cerebral aneurysm to limit blood flow within the aneurysm and to promote embolic formation.

Typically, the surgeon first forms a framework within the aneurysm by using a rigid coil, and then selects a flexible coil to fill the space within the framework. Ideally, each coil would be compliant with both the aneurysm and the pre-implanted coil.

During implantation, the surgeon manipulates each embolic coil until it is placed in a satisfactory position as observed by an imaging technique, such as fluoroscopic visualization, before detaching the coil from the delivery system. It is highly desirable that both ends of each coil remain positioned and maintained within the aneurysm after delivery, which is because a coil of a predetermined length protruding into the main lumen of a blood vessel results in undesirable clotting outside the aneurysm.

In addition, it is important to design the embolic coil to be anchored so as not to leave an intended site after being inserted into the site or to prevent the embolic coil from being released due to the pressure of blood flow.

However, the conventional embolic coils disclosed in U.S. Pat. Nos. 5,964,797 and 8,152,839 are problematic in that the anchoring function may not be properly performed or the embolic coil may be released due to the pressure of blood flow.

### DESCRIPTION OF EMBODIMENTS

### Technical Problem

One embodiment of the present invention provides an anchor for an embolic coil capable of improving the lesion treatment effect of the embolic coil.

Another embodiment of the present invention provides an embolic coil comprising the anchor for the embolic coil.

### Solution to Problem

An aspect of the present invention provides an anchor for an embolic coil comprising:

a hollow main body; and

a central shaft arranged in the hollow main body,

wherein the hollow main body is made of a braided composite filament comprising a polymer filament and a first alloy filament, and

the central shaft includes a second alloy filament that is a material different from the first alloy filament.

The hollow main body may have a network structure.

The polymer filament may include monofilaments, multifilaments, or a combination thereof.

The polymer filament may include polyester, polyamide, polyacrylonitrile, polyethylene, polybutylene, polyurethane, copolymers thereof, or combinations thereof.

The first alloy filament may include a platinum/tungsten wire.

The second alloy filament may include a shape memory alloy.

The second alloy filament may include nitinol.

The hollow main body may have a cylindrical shape with one end and the other end open, and the anchor for the embolic coil may further include an end cap sealing at least one of the one end and the other end.

The hollow main body may have one end open and the other end coupled to the central shaft and then stretched in one direction to be self-sealed, and the anchor for the embolic coil may further include an end cap for sealing the open end of the hollow main body.

The end cap may include platinum, polycarbonate, polyvinyl alcohol, or a combination thereof.

The central shaft may be configured to be coupled to the end cap.

The central shaft may be configured so as not to be coupled to the end cap.

Another aspect of the present invention provides an embolic coil comprising:

an anchor for the embolic coil; and

a primary coil coupled to the anchor for the embolic coil.

The embolic coil may further include a linker disposed between the anchor for the embolic coil and the primary coil to bind them to each other.

The linker may include a first hub coupled to one end of the anchor for the embolic coil, a second hub coupled to one end of the primary coil, and a luer lock disposed between the first hub and the second hub to couple the first and second hubs to each other.

Each of the first hub and the second hub may have a male thread formed on the outer peripheral surface thereof, and the luer lock may have female threads screw-joined with the male threads on one side and the other side, respectively.

The luer lock includes a luer lock female portion and a luer lock male portion,

the luer lock female portion includes a cylindrical main body with one end closed and the other end open, a first female thread formed on one side, a second female thread formed on the other side, and a protrusion disposed inside the main body and having one end coupled to the main body and the other end being a free end,

the luer lock male portion includes a cylindrical main body with at least one end open, a male thread formed on one side, and a third female thread formed on the other side,

the first female thread formed in the luer lock female portion is screw-joined with the male thread formed on the first hub, the second female thread formed on the luer lock female portion is screw-joined with the male thread formed on the luer lock male portion, the protrusion formed in the luer lock female portion is inserted into the hollow formed in the luer lock male portion, and

the third female thread formed in the luer lock male portion is screw-joined with the male thread formed on the second hub.

The linker may include a hook structure coupled to the end sealing portion of the anchor for the embolic coil and a ring structure coupled to one end of the primary coil, wherein the hook structure and the ring structure may be configured to be coupled to each other.

Another aspect of the present invention provides an embolic coil comprising:

a primary coil;

an anchor for a first embolic coil coupled to one end of the primary coil; and

an anchor for a second embolic coil coupled to the other end of the primary coil.

### Advantageous Effects of Disclosure

The anchor for an embolic coil according to an embodiment of the present invention can improve anchoring characteristics, coil retention characteristics, and thrombus collection characteristics of the embolic coil in a blood vessel.

### BRIEF DESCRIPTION OF DRAWINGS

FIG. 1 is a view schematically illustrating an anchor for an embolic coil, according to an embodiment of the present invention.

FIG. 2 is an enlarged photograph showing a braided composite filament constituting a hollow main body of the anchor for an embolic coil shown in FIG. 1.

FIG. 3 is a view schematically illustrating a first embodiment of an embolic coil comprising the anchor for an embolic coil shown in FIG. 1.

FIG. 4 is an enlarged view of a portion of a linker in the embolic coil of FIG. 2.

FIG. 5 is a view for explaining a second embodiment of the embolic coil comprising the anchor for a embolic coil shown in FIG. 1, and a method for using same.

### BEST MODE

Hereinafter, an anchor for an embolic coil and an embolic coil, according to an embodiment of the present invention, will be described in detail with reference to the drawings.

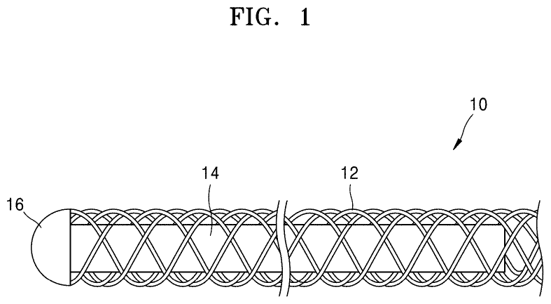

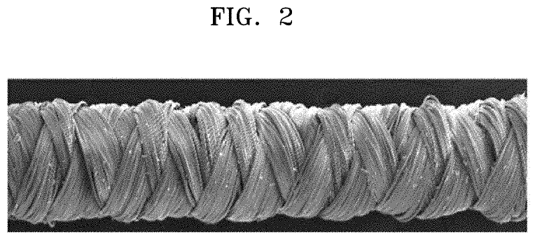

FIG. 1 is a view schematically illustrating an anchor 10 for an embolic coil according to an embodiment of the present invention, and FIG. 2 is an enlarged photograph showing a braided composite filament constituting a hollow main body 12 of the anchor 10 for an embolic coil shown in FIG. 1.

Referring to FIG. 1, the anchor 10 for an embolic coil according to an embodiment of the present invention includes a hollow main body 12 and a central shaft 14.

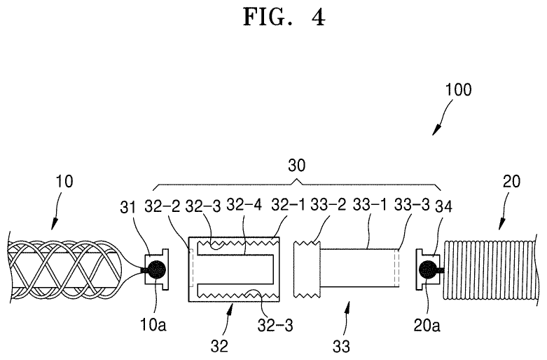

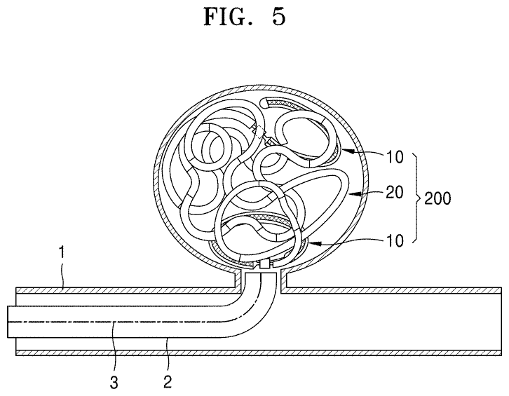

When the anchor 10 for an embolic coil is inserted into an abnormal blood vessel (that is, a blood vessel swollen in the alveolar shape) and is exposed to warm blood, the central shaft 14, the transformation temperature of which is set in a human body temperature range (30 to 45° C.), may be bent, and the hollow main body 12 may also be bent accordingly, thereby anchoring the embolic coil (100 of FIGS. 2 and 4 and/or 200 of FIG. 5) including the same to the inner wall of a blood vessel, or supporting the embolic coil so as to prevent even a portion of the embolic coil from escaping from the abnormal blood vessel.

In addition, the anchor 10 for an embolic coil serves to collect thrombus from blood.

The hollow main body 12 may be made of a braided composite filament including a polymer filament and a first metal alloy filament. As shown in FIG. 2, the braided composite filament may be formed by braiding a filament bundle made of one or more polymer filaments and one or more first alloy filaments together.

The hollow main body 12 may have excellent thrombus trapping properties due to the polymer filament having superior thrombus adhesion properties compared to conventional alloy filaments.

In addition, the hollow main body 12 may have high durability due to the braided composite filament having excellent toughness and rigidity, strong resistance to damage due to external force, and difficult to unwind even when exposed to blood pressure.

The hollow main body 12 may have a network structure. Specifically, the hollow main body 12 may be manufactured by weaving the braided composite filament into a network structure. Accordingly, the hollow main body 12 may have fluid permeability. The fluid permeability means blood and gas permeability.

The polymer filament may include a monofilament, multifilaments, or combinations thereof (e.g., one or two thereof is (are) made of a combination of one or more strands).

In addition, the polymer filament may include polyester, polyamide, polyacrylonitrile, polyethylene, polybutylene, polyurethane, a copolymer thereof, or a combination thereof (e.g., one or two thereof is (are) made of a combination of one or more strands).

The polyester may include polyglycolic acid, polylactic acid, polycaprolactone, polyhydroxyalkanoate, polyhydroxybutyrate, polyethylene adipate, polybutylene succinate, poly(3-hydroxybutyrate-co-3-hydroxyvalerate), polyethylene terephthalate, polybutylene terephthalate, polytrimethylene terephthalate, polyethylene naphthalate, Vectran, or a combination thereof.

The polyamide may include aliphatic polyamide, polyphthalamide, aramid, or a combination thereof.

The copolymer may be a copolymer of two or more monomers selected from a monomer for polymerizing the polyester, a monomer for polymerizing the polyamide, a monomer for polymerizing the polyacrylonitrile, a monomer for polymerizing the polyethylene, a monomer for polymerizing the polybutylene, and a monomer for polymerizing the polyurethane.

The first alloy filament may include a platinum/tungsten wire (e.g., one or two thereof is (are) made of a combination of one or more strands).

The first alloy filament may have a diameter of about 10 μm to about 100 μm (e.g., 50 μm).

The central shaft 14 may be arranged in the hollow main body 12. The central shaft 14 may have a straight shape extending in one direction.

In addition, the central shaft 14 may include a second alloy filament that is a material different from the first alloy. In the present specification, the phrase “the second alloy filament that is a material different from the first alloy filament” means that the first alloy filament and the second alloy filament have one or more different physical properties according to the types of components, the content ratios of the components, and heat treatment conditions.

The second alloy filament may include a shape memory alloy. For example, the second alloy filament may be a material which maintains the original shape thereof without a change in shape when exposed to room temperature (about 25° C.) that is an outside human body temperature while shrinking into a hook shape at high temperature (30° C. to 45° C.) inside a human body, and is restored to the original shape thereof when exposed to room temperature again. Accordingly, the central shaft 14 including the second alloy filament, and the anchor 10, may also exhibit the same or similar shape change as the shape change of the second alloy filament. In detail, the central shaft 14 including the second alloy filament, and the anchor 10, may maintain the straight shape extending in one direction when exposed to room temperature (about 25° C.) that is an outside human body temperature while shrinking into a hook shape at high temperature (30° C. to 45° C.) inside a human body, and is restored to the original straight shape thereof when exposed to room temperature again.

The second alloy filament may include nitinol. In the present specification, the term “nitinol” refers to an alloy of nickel and titanium, and means an alloy having a nickel:titanium atomic percentage of about 50:50. For example, the second alloy filament may include nitinol 55 or nitinol 60.

The second alloy filament may have a diameter of 10 μm to 300 μm (e.g., 50 μm).

The anchor 10 for an embolic coil may further include one or more end caps 16.

The end cap 16 may include platinum, polycarbonate, polyvinyl alcohol, or a combination thereof.

As an example, although not shown in the figures, the hollow main body 12 may have a cylindrical shape with one end and the other end open. In this case, the hollow main body 12 may include one or two end caps 16 sealing at least one of the one end and the other end that are open. In addition, in this case, the central shaft 14 may have at least one of one end and the other end thereof coupled to the one or two end caps 16, and both of the one end and the other end may be disposed in a state in which they are not coupled to the end caps 16.

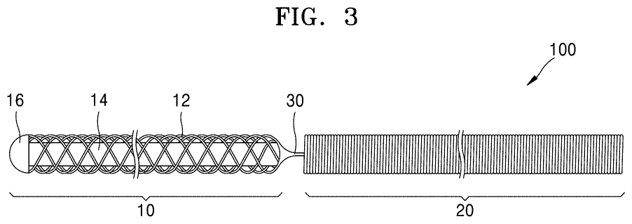

As another example, as shown in FIGS. 3 and 4, the hollow main body 12 may have one end open and the other end coupled to the central shaft 14 and then stretched in one direction to be self-sealed. For example, the hollow main body 12 may have one end open and the other end coupled to the central shaft 14 and then stretched in one direction with an end of the central shaft 14 to be self-sealed. In this case, the hollow main body 12 may include one end cap 16 for sealing the open end thereof. In addition, in this case, the central shaft 14 may be disposed in a state in which one end thereof is coupled to the end cap 16 or a state in which one end thereof is not coupled to the end cap 16.

FIG. 3 is a view schematically illustrating a first embodiment of the embolic coil 100 comprising the anchor 10 for an embolic coil shown in FIG. 1, and FIG. 4 is an enlarged view of a portion of a linker 30 in the embolic coil 100 of FIG. 2.

Referring to FIGS. 3 and 4, the embolic coil 100 according to a first embodiment of the present invention includes an anchor 10 for an embolic coil and a primary coil 20. For example, the embolic coil 100 may include one or more anchors 10 for embolic coils and one or more primary coils 20.

The embolic coil 100 is inserted into an abnormal blood vessel to block the abnormal blood vessel so as to prevent blood from flowing into the abnormal blood vessel, while collecting thrombus from the blood that flows into a normal blood vessel.

Since the anchor 10 for an embolic coil has been described in detail above, a detailed description thereof will be omitted.

When the embolic coil 100 including the primary coil 20 is inserted into an abnormal blood vessel, the primary coil 20 fills the inside of the abnormal blood vessel and blocks the abnormal blood vessel to prevent blood from flowing into the abnormal blood vessel. To this end, the primary coil 20 may be configured to have, for example, a dense coil shape or a spring shape so as to have a higher packing density than the anchor 10 in the abnormal blood vessel.

In addition, the primary coil 20 may have better ductility than the anchor 10 for an embolic coil.

In addition, the primary coil 20 may have a wound ribbon shape.

In addition, the primary coil 20 may include a third alloy filament, and the third alloy filament may include a platinum/tungsten wire.

The embolic coil 100 may further include a linker 30.

The linker 30 is disposed between the anchor 10 and the primary coil 20 and serves to bind the anchor 10 and the primary coil 20 to each other.

In addition, the linker 30 may have one end coupled to a thinly stretched portion of the anchor 10 and the other end coupled to a thin strand of the third alloy filament extending from the primary coil 20. Therefore, the linker 30 may minimize the diameter and length of the region where the anchor 10 and the primary coil 20 are coupled.

In addition, referring to FIG. 4, the linker 30 may include a first hub 31, a second hub 34, and luer locks 32 and 33.

The first hub 31 may be coupled to one end 10a (e.g., an end stretched in one direction) of the anchor 10.

In addition, the first hub 31 may include a male thread (not shown) formed on an outer peripheral surface thereof.

The second hub 34 may be coupled to one end 20a of the primary coil (e.g., a strand of the third alloy filament).

In addition, the second hub 34 may include a male thread (not shown) formed on an outer peripheral surface thereof.

The luer locks 32 and 33 may be disposed between the first hub 31 and the second hub 34 and may serve to couple the first hub 31 and the second hub 34 to each other.

The luer lock 32, 33 may include a female thread 32-2 screw-joined with the male thread of the first hub 31 on one side and a female thread 33-3 screw-joined with the male thread of the second hub 34 on the other side.

The luer locks 32 and 33 may include a luer lock female portion 32 and a luer lock male portion 33.

The luer lock female portion 32 may include a main body 32-1, a first female thread 32-2, a second female thread 32-3, and a protrusion 32-4.

The main body 32-1 may have a cylindrical shape with one end closed and the other end open.

The first female thread 32-2 may be formed on one side of the main body 32-1. The first female thread 32-2 may be screw-joined with the male thread formed on the first hub 31.

The second female thread 32-3 may be formed on the other side of the main body 32-1. This second female thread 32-3 may be screw-joined with a male thread 33-2 formed in the luer lock male portion 33, which will later be described.

The protrusion 32-4 may be disposed inside the main body 32-1, and one end thereof may be coupled to the main body 32-1, and the other end may be a free end. The protrusion 32-4 may have a straight shape extending in one direction as a whole. Accordingly, the protrusion 32-4 may be inserted into the hollow of the main body 33-1 formed in the luer lock male portion 33, which will later be described. As described above, since the protrusion 32-4 of the luer lock female portion 32 is inserted into the hollow of the luer lock male portion 33, the luer lock female portion 32 and the luer lock male portion 33 can be stably coupled to each other without shaking.

The luer lock male portion 33 may include a main body 33-1, a male thread 33-2, and a third female thread 33-3.

The main body 33-1 may have a cylindrical shape with at least one end open. Accordingly, the main body 33-1 may have a hollow.

The male thread 33-2 may be formed on one side of the main body 33-1.

The third female thread 33-3 may be formed on the other side of the main body 33-1. The third female thread 33-3 may be screw-joined with the male thread formed on the second hub 34.

The linker may have various structures other than the above-described luer lock type linker. For example, although not shown, the linker may include a hook structure coupled to an end sealing portion (which is formed by collecting one-end alloy filaments (that is, the hollow main body (12)) of the anchor and end-sealing the same), and a ring structure coupled to one end of the primary coil, wherein the hook structure and the ring structure are configured to be coupled to each other.

FIG. 5 is a view for explaining a second embodiment of the embolic coil 200 comprising the anchor 10 for an embolic coil shown in FIG. 1, and a method for using same.

Referring to FIG. 5, the embolic coil 200 according to the second embodiment of the present invention includes a primary coil 20 and two anchors 10 for embolic coils.

One of the two anchors 10 for embolic coils (i.e., a first anchor for an embolic coil) may be coupled to one end of the primary coil 20, and the other one (i.e., a second anchor for an embolic coil) may be coupled to the other end of the primary coil 20.

Hereinafter, a method for inserting the embolic coil 200 into an abnormal blood vessel (that is, a blood vessel swollen in an alveolar shape) will be described.

First, a micro-conduit 2 such as a catheter is inserted from a normal blood vessel 1 to the entrance of an abnormal blood vessel.

Thereafter, the embolic coil 200 is inserted into the micro-conduit 2.

Next, the embolic coil 200 is pushed with a pusher wire 3 to insert the embolic coil 200 into the abnormal blood vessel.

In the embolic coil 200 inserted into the abnormal blood vessel, the anchor 10 located on the wall side of the abnormal blood vessel acts as an anchor, and another anchor 10 located at the entrance side of the abnormal blood vessel performs a support function, thereby allowing the embolic coil 200 to be fixed in position in the abnormal blood vessel without deviating from the abnormal blood vessel. Accordingly, the embolic coil 200 (specifically, the primary coil 20) is not exposed to the blood flow of a normal blood vessel 1, and thus can be prevented from being released due to the pressure of the blood flow.

While one or more exemplary embodiments have been described with reference to the figures, the embodiments described herein have been presented by way of example only, and it will be appreciated by those skilled in the art that various changes and other equivalent embodiments may be made from the above description. Therefore, the true technical protection scope of the present invention should be defined by the inventive concept of the appended claims.

1. An anchor for an embolic coil, comprising:
a hollow main body; and
a central shaft arranged in the hollow main body,
wherein the hollow main body is made of a braided composite filament comprising a polymer filament and a first alloy filament, and
the central shaft includes a second alloy filament that is a material different from the first alloy filament.

2. The anchor of claim 1, wherein the hollow main body has a network structure.

3. The anchor of claim 1, wherein the polymer filament includes monofilaments, multifilaments, or a combination thereof.

4. The anchor of claim 1, wherein the polymer filament includes polyester, polyamide, polyacrylonitrile, polyethylene, polybutylene, polyurethane, a copolymer thereof, or a combination thereof.

5. The anchor of claim 1, wherein the first alloy filament includes a platinum/tungsten wire.

6. The anchor of claim 1, wherein the second alloy filament includes a shape memory alloy.

7. The anchor of claim 6, wherein the second alloy filament includes nitinol.

8. The anchor of claim 1, wherein the hollow main body has a cylindrical shape with one end and the other end open, and the anchor further comprises an end cap for sealing at least one of the one end and the other end.

9. The anchor of claim 1, wherein the hollow main body has one end open and the other end coupled to the central shaft and then stretched in one direction to be self-sealed, and the anchor further comprises an end cap for sealing the open end of the hollow main body.

10. The anchor of claim 8 or 9, wherein the end cap includes platinum, polycarbonate, polyvinyl alcohol, or a combination thereof.

11. The anchor of claim 8, wherein the central shaft is configured to be coupled to the end cap.

12. The anchor of claim 8, wherein the central shaft is configured so as not to be coupled to the end cap.

13. An embolic coil comprising:
the anchor for the embolic coil of claim 1; and
a primary coil coupled to the anchor for the embolic coil.

14. The embolic coil of claim 13, further comprising a linker disposed between the anchor for the embolic coil and the primary coil to bind the embolic coil and the primary coil to each other.

15. The embolic coil of claim 14, wherein the linker comprises:
a first hub coupled to one end of the anchor for the embolic coil;
a second hub coupled to one end of the primary coil; and
a luer lock disposed between the first hub and the second hub to couple the first and second hubs to each other.

16. The embolic coil of claim 15, wherein each of the first hub and the second hub has a male thread formed on an outer peripheral surface thereof, and the luer lock has female threads screw-joined with the male threads on one side and the other side, respectively.

17. The embolic coil of claim 16, wherein
the luer lock includes a luer lock female portion and a luer lock male portion,
the luer lock female portion includes a cylindrical main body with one end closed and the other end open, a first female thread formed on one side, a second female thread formed on the other side, and a protrusion disposed inside the main body and having one end coupled to the main body and the other end being a free end,
the luer lock male portion includes a cylindrical main body with at least one end open, a male thread formed on one side, and a third female thread formed on the other side,
the first female thread formed on the luer lock female portion is screw-joined with the male thread formed on the first hub, the second female thread formed on the luer lock female portion is screw-joined with the male thread formed on the luer lock male portion, the protrusion formed on the luer lock female portion is inserted into a hollow formed in the luer lock male portion, and
the third female thread formed on the luer lock male portion is screw-joined with the male thread formed on the second hub.

18. The embolic coil of claim 14, wherein the linker comprises:
a hook structure coupled to an end sealing portion of the anchor for the embolic coil; and
a ring structure coupled to one end of the primary coil, wherein
the hook structure and the ring structure are configured to be coupled to each other.

19. An embolic coil comprising:
a primary coil;
the anchor for the embolic coil of claim 1 coupled to one end of the primary coil; and
the anchor for the embolic coil of claim 1 coupled to the other end of the primary coil.
